(19) United States
(12) Patent Application Publication
(10) Pub. No.: US 2022/0072075 A1
(43) Pub. Date: Mar. 10, 2022

(54) METHOD FOR PRODUCING NATURAL ANTIMICROBIAL SUBSTANCE AND ANTIMICROBIAL COMPOSITION COMPRISING NATURAL ANTIMICROBIAL SUBSTANCE PRODUCED BY THE SAME

(71) Applicant: MY CO.,LTD, Gwangju-si (KR)

(72) Inventors: Pyo SONG, Seoul (KR); Sun Lin KIM, Sungnamsi (KR)

(21) Appl. No.: 17/116,337

(22) Filed: Dec. 9, 2020

(30) Foreign Application Priority Data
Sep. 4, 2020 (KR) 10-2020-0113090

(51) Int. Cl. A61K 36/235; A61K 36/704; A61K 47/10

(52) U.S. Cl. CPC A61K 36/235; A61K 47/10; A61K 36/704

(57) ABSTRACT

A natural antimicrobial substance produced by applying electrical stimulation to a nano-sized Foeniculum vulgare extract and a nano-sized Polygonum tinctorium extract, and an antimicrobial composition produced by adding a sugar alcohol to the natural antimicrobial substance. The antimicrobial composition has advantages in that it is safe for human cell membranes and, at the same time, exhibits maximized antimicrobial efficacy by disrupting the cell membrane structure of bacteria.

### CROSS-REFERENCE TO RELATED APPLICATION

This application claims priority to and the benefit of Korean Patent Application No. 10-2020-0113090, filed on Sep. 4, 2020, the disclosure of which is incorporated herein by reference in its entirety.

### BACKGROUND

### 1. Technical Field

The present disclosure relates to a method for producing a natural antimicrobial substance and an antimicrobial composition comprising a natural antimicrobial substance produced by the method.

### 2. Related Art

Antimicrobial substances are agents that can prevent the growth of microorganisms and are used to remove bacteria, fungi or viruses.

About 130 kinds of antimicrobial agents have been developed to date. These antimicrobial agents may be classified into synthetic antimicrobial agents and natural antimicrobial agents, and the market size of the synthetic antimicrobial agents is 5 to 6 times larger than that of the natural antimicrobial agents.

Currently, the use of synthetic antimicrobial agents is limited due to the toxicity thereof. Due to this shortcoming of the synthetic antimicrobial agents, efforts have been actively made to replace the synthetic antimicrobial agents with natural antimicrobial agents.

### PRIOR ART DOCUMENTS

### Patent Documents

Korean Patent No. 2124592 (registered on Jun. 12, 2020), entitled “Hand Sanitizer Composition Having Antibacterial and Antiviral Activities, Containing Natural Extracts”.

### SUMMARY

An object of the present disclosure is to provide an antimicrobial substance extracted from natural products.

A method for producing a natural antimicrobial substance according to one embodiment of the present disclosure may comprise steps of: adding an organic solvent to each of a Foeniculum vulgare extract aqueous solution and a Polygonum tinctorium extract aqueous solution to obtain a Foeniculum vulgare extract fraction and a Polygonum tinctorium extract fraction; mixing the Foeniculum vulgare extract fraction and the Polygonum tinctorium extract fraction to obtain a Foeniculum vulgare extract fraction/Polygonum tinctorium extract fraction mixture; applying ultrasonic stimulation to the obtained Foeniculum vulgare extract fraction/Polygonum tinctorium extract fraction mixture to obtain a nano-sized Foeniculum vulgare extract fraction/Polygonum tinctorium extract fraction mixture; and applying electrical stimulation to the nano-sized Foeniculum vulgare extract fraction/Polygonum tinctorium extract fraction mixture.

In the present invention, the weight ratio between the Foeniculum vulgare extract aqueous solution, the Polygonum tinctorium extract aqueous solution and the organic solvent may be 1:1.5:7 to 1:2:8.

An antimicrobial composition according to one embodiment of the present disclosure may comprise: a natural antimicrobial substance produced by the above-described method; and a sugar alcohol.

In addition, the antimicrobial composition may comprise 10 to 30 parts by weight of the Foeniculum vulgare extract, 20 to 30 parts by weight of the Polygonum tinctorium extract, 10 to 20 parts by weight of sorbitol, and 20 to 40 parts by weight of maltitol.

A disinfectant for non-human use according to one embodiment of the present disclosure may be obtained by adding the antimicrobial composition to multi-photocatalyst water or hypochlorous acid water (pH 2.7 to 6.5) at a concentration of 0.1 to 5%.

A disinfectant for human use according to one embodiment of the present disclosure may be obtained by adding the antimicrobial composition to multi-photocatalyst water or hypochlorous acid water (pH 2.7 to 6.5) at a concentration of 0.1 to 0.5%.

### BRIEF DESCRIPTION OF THE DRAWINGS

FIGS. 1A to 1F are photographs of solid culture dishes in which methicillin-resistant Staphylococcus aureus (MRSA) mixed with different concentrations of a natural antimicrobial substance has been incubated.

FIGS. 2A to 2F are photographs of solid culture dishes in which carbapenemase-producing Enterobacteriaceae (CPE) mixed with different concentrations of a natural antimicrobial substance has been incubated.

FIG. 3 shows the results of testing whether a natural antimicrobial substance (1%) according to one embodiment of the present disclosure exhibits sustained antimicrobial activity against MRSA.

FIG. 4 shows the data obtained by identifying Staphylococcus aureus and Staphylococcus aureus plus distilled water using a mass spectrometer.

FIG. 5 shows the data obtained by identifying Staphylococcus aureus and Staphylococcus aureus plus 70% ethanol using a mass spectrometer.

FIG. 6 shows the data obtained by identifying Staphylococcus aureus and Staphylococcus aureus plus Febreze™ using a mass spectrometer.

FIG. 7 shows the data obtained by identifying Staphylococcus aureus and Staphylococcus aureus plus the natural antimicrobial substance (0.1%) of the present disclosure using a mass spectrometer.

FIG. 8 shows the data obtained by identifying Staphylococcus aureus and Staphylococcus aureus plus the natural antimicrobial substance (0.5%) of the present disclosure using a mass spectrometer.

FIG. 9 shows the data obtained by identifying Staphylococcus aureus and Staphylococcus aureus plus the natural antimicrobial substance (1%) of the present disclosure using a mass spectrometer.

FIG. 10 shows the data obtained by identifying Pseudomonas aeruginosa and Pseudomonas aeruginosa plus the natural antimicrobial substance (0.05%) of the present disclosure using a mass spectrometer.

FIG. 11 shows the data obtained by identifying Pseudomonas aeruginosa and Pseudomonas aeruginosa plus the natural antimicrobial substance (0.1%) of the present disclosure using a mass spectrometer.

FIG. 12 shows the data obtained by identifying Pseudomonas aeruginosa and Pseudomonas aeruginosa plus the natural antimicrobial substance (0.5%) of the present disclosure using a mass spectrometer.

FIG. 13 shows the data obtained by identifying Pseudomonas aeruginosa and Pseudomonas aeruginosa plus the natural antimicrobial substance (1%) of the present disclosure using a mass spectrometer.

### DETAILED DESCRIPTION

Hereinafter, the present disclosure will be described in detail.

Method for Producing Natural Antimicrobial Substance

As used herein, the term “antimicrobial” means not only removing germs, but also removing any one of fungi, viruses, parasites and bacteria. Therefore, the term “antimicrobial agent” as used herein refers to a substance capable of removing any one of germs, fungi, viruses and bacteria, and is not necessarily limited to the dictionary definition “antimicrobial”.

A method for producing a natural antimicrobial substance according to one embodiment of the present disclosure may comprise steps of: (S10) fermenting washed and screened natural plant components, followed by extraction under reflux with an extraction solvent to obtain extracts; (S20) filtering, concentrating and freeze-drying each of the extracts to obtain powders; (S30) dispersing each of the powders in distilled water, followed by fractionation with an organic solvent to obtain fractions; (S40) mixing the fractions to obtain a fraction mixture, and applying ultrasonic stimulation to the fraction mixture to obtain a nano-sized fraction mixture; (S50) applying electrical stimulation to the nano-sized fraction mixture so that the nano-sized natural plant components penetrate each other; and (S60) heating the nano-sized fraction mixture to which the electrical stimulation has been applied, followed by vacuum drying.

1. Step (S10) of Obtaining Extracts

Foeniculum vulgare and Polygonum tinctorium are washed clean and are separately extracted twice with ethanol as an extraction solvent for 1 hour each to obtain a Foeniculum vulgare extract and a Polygonum tinctorium extract.

2. Step (S20) of Powdering

Next, each of the Foeniculum vulgare extract and the Polygonum tinctorium extract is filtered by a conventional method. The filtered Foeniculum vulgare extract and Polygonum tinctorium extract are concentrated by a rotary evaporator. The concentrated Foeniculum vulgare extract and Polygonum tinctorium extract are separately freeze-dried to obtain powders.

3. Step (S30) of Obtaining Extract Fractions

Next, each of the Foeniculum vulgare extract powder and the Polygonum tinctorium extract powder is mixed with and dispersed in distilled water, and an organic solvent is added thereto to obtain a Foeniculum vulgare extract fraction and a Polygonum tinctorium extract fraction. Here, the organic solvent is preferably one or more selected from among hexane, chloroform, ethyl acetate, and butanol. The Foeniculum vulgare extract fraction and the Polygonum tinctorium extract fraction are mixed together to obtain a Foeniculum vulgare extract fraction/Polygonum tinctorium extract fraction mixture.

Preferably, the weight ratio between the Foeniculum vulgare extract powder and distilled water is the same as the weight ratio between the Polygonum tinctorium extract powder and distilled water. Preferably, the weight ratio between each powder and distilled water may be 1:3 to 1:4.

In addition, the weight ratio between the Foeniculum vulgare extract powder dispersed in distilled water (hereinafter referred to as “Foeniculum vulgare extract aqueous solution”), the Polygonum tinctorium extract powder dispersed in distilled water (hereinafter referred to as “Polygonum tinctorium extract aqueous solution”), and the organic solvent may be 1:1.5:7 to 1:2:8.

Preferably, 20 parts by weight of the Foeniculum vulgare extract aqueous solution, 30 parts by weight of the Polygonum tinctorium extract aqueous solution and 150 parts by weight of the organic solvent may be mixed together and then vortexed for 3 to 5 minutes.

4. Step (S40) of Obtaining Nano-Sized Mixture

Next, ultrasonic stimulation is applied to the Foeniculum vulgare extract fraction/Polygonum tinctorium extract fraction mixture to obtain a nano-sized mixture. Application of the ultrasonic stimulation may be performed by stimulating the fraction mixture with ultrasonic waves at a frequency of 35,000 Hz to 50,000 Hz with 3 minutes and then stabilizing the fraction mixture for 2 minutes using a liposofast (Avestin, Canada) nano-extruder, and repeating this stimulation and stabilization process a total of 12 times. Thereby, a nano-sized Foeniculum vulgare extract fraction/Polygonum tinctorium extract fraction mixture may be obtained.

5. Step (S50) of Applying Electrical Stimulation

Thereafter, electrical stimulation is applied to the extract fraction mixture so that the nano-sized Foeniculum vulgare extract fraction penetrates the nano-sized Polygonum tinctorium extract fraction. Here, the electrical stimulation may be performed once at a voltage of 80 to 110 V and at a current of 50 to 65 mA for 10 to 30 minutes or performed twice at a voltage of 50 to 110V and at a current of 30 to 50 mA for 10 to 20 minutes. In this electrical stimulation process, the nano-sized Foeniculum vulgare extract fraction may penetrate the nano-sized Polygonum tinctorium extract fraction, or the nano-sized Polygonum tinctorium extract fraction may penetrate the nano-sized Foeniculum vulgare extract fraction.

6. Step (S60) of Heating and Vacuum-Drying Mixture

Next, the nano-sized Foeniculum vulgare extract fraction/Polygonum tinctorium extract fraction mixture, to which electrical stimulation has been applied, may be heated at a temperature of 900° C. to 1,100° C. for 25 to 35 minutes and then vacuum-dried for 7 hours to obtain a natural antimicrobial substance.

The natural antimicrobial substance comprises the Foeniculum vulgare extract and the Polygonum tinctorium extract. Preferably, the weight ratio between the Foeniculum vulgare extract and the Polygonum tinctorium extract may be 1:0.1 to 1:5.

The above-described natural antimicrobial substance may have maximized antimicrobial efficacy because the nano-sized Foeniculum vulgare extract and the nano-sized Polygonum tinctorium extract penetrate each other by an ionic reaction to form a special structure that disrupts the cell membrane structure of bacteria. In addition, the special structure has the advantage of being safe for human cell membranes.

Antimicrobial Composition Comprising Natural Antimicrobial Substance

An antimicrobial composition according to one embodiment of the present disclosure may comprise the natural antimicrobial substance and a sugar alcohol. The sugar alcohol may comprise at least one of sorbitol, xylitol, mannitol, erythritol, and lactitol.

The antimicrobial composition comprising the natural antimicrobial substance according to one embodiment of the present disclosure may comprise the Foeniculum vulgare extract, the Polygonum tinctorium extract, sorbitol, and maltitol.

In one embodiment, the antimicrobial composition may comprise 0.1 to 30 parts by weight of the Foeniculum vulgare extract, 2 to 30 parts by weight of the Polygonum tinctorium extract, 4 to 20 parts by weight of sorbitol, and 5 to 40 parts by weight of maltitol.

Preferably, the antimicrobial composition may comprise 1 to 30 parts by weight of the Foeniculum vulgare extract, 5 to 30 parts by weight of the Polygonum tinctorium extract, 10 to 20 parts by weight of sorbitol, and 20 to 40 parts by weight of maltitol.

The Foeniculum vulgare extract and the Polygonum tinctorium extract are produced by the above-described method for producing a natural antimicrobial substance. The nano-sized Foeniculum vulgare extract and the nano-sized Polygonum tinctorium extract may penetrate each other to increase binding affinity between active ingredients having antimicrobial activity, thereby increasing antimicrobial function.

Maltitol and sorbitol that are used in the present disclosure may kill bacteria by interfering with the metabolism of the bacteria. In addition, maltitol may be converted into mannose, and sorbitol may be converted into glycans such as glucose. The resulting glycans are constituent compounds surrounding bacteria and viruses, and may affect the interaction between the bacteria and the bacteria or the viruses and the host, thereby inhibiting the activities of the bacteria and viruses.

In addition, sorbitol and maltitol have the advantage of being harmless to the human body so that they are allowed as food additives in Korea.

Antimicrobial Efficacy Test 1

(1) Methicillin-resistant Staphylococcus aureus (MRSA), an antibiotic-resistant superbacterium, was inoculated into a liquid medium (S1).

(2) MRSA inoculated into the liquid medium was mixed with various concentrations of an antimicrobial composition (referred to as NX2020) comprising the above-described antimicrobial substance, and each of the mixtures was incubated in an incubator at 37° C. for 12 hours (S2).

Next, (3) 50 μl of the liquid medium incubated at each concentration for 12 hours was plated on each solid culture dish (BAP medium) (S3).

Thereafter, (4) each liquid medium was incubated in an incubator at 37° C. for 6 hours or more (S4).

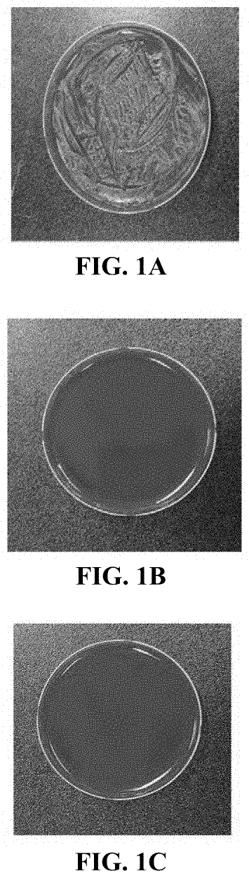

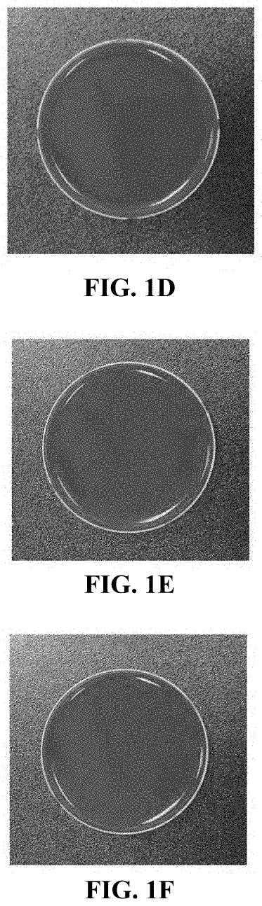

FIG. 1A is a photograph of a solid culture dish obtained by performing step S2 in which the liquid medium, which does not comprise the antimicrobial composition, is incubated in an incubator at 37° C. for 12 hours, and then performing steps S3 and S4.

FIG. 1B is a photograph of a solid culture dish obtained by performing step S2 in which the MRSA liquid medium is mixed with a solution of the antimicrobial composition (1% concentration) in multi-photocatalyst water and then incubated in an incubator at 37° C. for 12 hours, and then performing steps S3 and S4.

FIG. 1C is a photograph of a solid culture dish obtained by performing step S2 in which the MRSA liquid medium is mixed with a solution of the antimicrobial composition (0.5% concentration) in multi-photocatalyst water and then incubated in an incubator at 37° C. for 12 hours, and then performing steps S3 and S4.

FIG. 1D is a photograph of a solid culture dish obtained by performing step S2 in which the MRSA liquid medium is mixed with a solution of the antimicrobial composition (1% concentration) in hypochlorous acid water (pH 5) and then incubated in the incubator at 37° C. for 12 hours, and then performing steps S3 and S4.

FIG. 1E is a photograph of a solid culture dish obtained by performing step S2 in which the MRSA liquid medium is mixed with a solution of the antimicrobial composition (0.5% concentration) in hypochlorous acid water (pH 5) and then incubated in the incubator at 37° C. for 12 hours, and then performing steps S3 and S4.

FIG. 1F is a photograph of a solid culture dish as a control obtained by performing step S2 in which the MRSA liquid medium is mixed with a 1% solution of Febreze™ and then incubated in the incubator at 37° C. for 12 hours, and then performing steps S3 and S4.

FIGS. 1B to 1E all showed an antimicrobial efficacy of 99.9%.

Antimicrobial Efficacy Test 2

(1) Carbapenemase-producing Enterobacteriaceae (CPE), an antibiotic-resistant superbacterium, was inoculated into a liquid medium (S5).

(2) CPE inoculated into the liquid medium was mixed with various concentrations of an antimicrobial composition (referred to as NX2020) comprising the above-described antimicrobial substance, and each of the mixtures was incubated in an incubator at 37° C. for 12 hours (S6).

Next, (3) 50 μl of the CPE liquid medium incubated at each concentration for 12 hours was plated on each solid culture dish (BAP medium) (S7).

Thereafter, (4) each liquid medium was incubated in an incubator at 37° C. for 6 hours or more (S8).

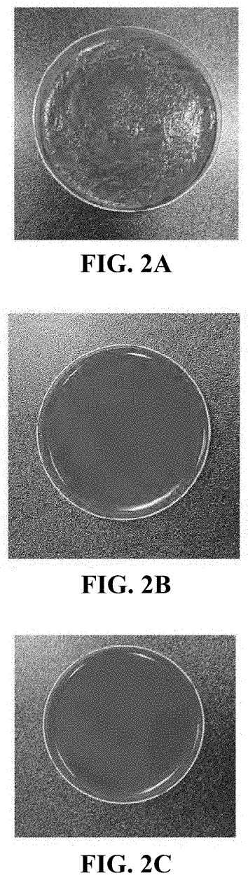

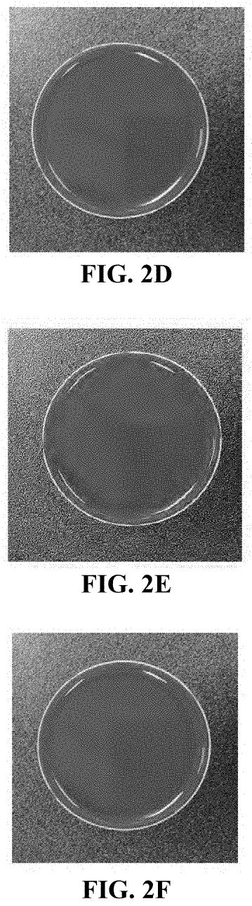

FIG. 2A is a photograph of a solid culture dish obtained by performing step S6 in which the liquid medium, which does not comprise the antimicrobial composition, is incubated in an incubator at 37° C. for 12 hours, and then performing steps S7 and S8.

FIG. 2B is a photograph of a solid culture dish obtained by performing step S6 in which the CPE liquid medium is mixed with a solution of the antimicrobial composition (1% concentration) in multi-photocatalyst water and then incubated in an incubator at 37° C. for 12 hours, and then performing steps S7 and S8.

FIG. 2C is a photograph of a solid culture dish obtained by performing step S6 in which the CPE liquid medium is mixed with a solution of the antimicrobial composition (0.5% concentration) in multi-photocatalyst water and then incubated in an incubator at 37° C. for 12 hours, and then performing steps S7 and S8.

FIG. 2D is a photograph of a solid culture dish obtained by performing step S6 in which the CPE liquid medium is mixed with a solution of the antimicrobial composition (1% concentration) in hypochlorous acid water (pH 5) and then incubated in the incubator at 37° C. for 12 hours, and then performing steps S7 and S8.

FIG. 2E is a photograph of a solid culture dish obtained by performing step S6 in which the CPE liquid medium is mixed with a solution of the antimicrobial composition (0.5% concentration) in hypochlorous acid water (pH 5) and then incubated in the incubator at 37° C. for 12 hours, and then performing steps S7 and S8.

FIG. 2F is a photograph of a solid culture dish as a control obtained by performing step S6 in which the CPE liquid medium is mixed with a 1% solution of Febreze™ and then incubated in the incubator at 37° C. for 12 hours, and then performing steps S7 and S8.

FIGS. 2B to 2E all showed an antimicrobial efficacy of 99.9%.

As used herein, the term “multi-photocatalyst water” refers to a solution obtained by adding water to a multi-photocatalyst. Here, the multi-photocatalyst causes a photocatalytic reaction regardless of the presence or absence of light, and may preferably comprise at least one of a titanium phosphate compound, a titanium dioxide compound, titanium, manganese, and manganese dioxide. When a multi-photocatalyst is added to water, active oxygen is be generated in the water, which can remove microorganisms harmful to the human body, such as bacteria, fungi, and viruses.

Antimicrobial Efficacy Test 3

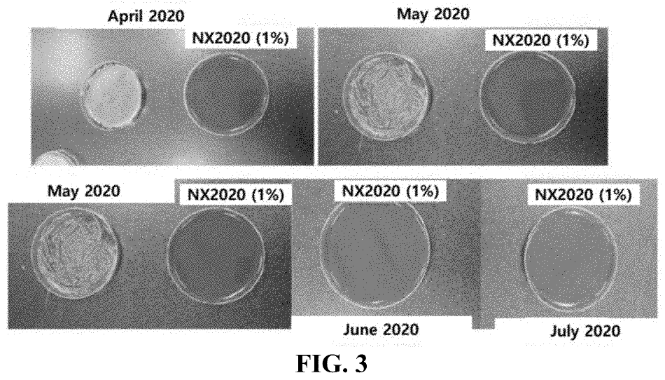

FIG. 3 shows the results of testing whether a natural antimicrobial substance (1%) according to one embodiment of the present disclosure has sustained antimicrobial activity against MRSA. Referring to FIG. 3, it can be seen that the antimicrobial activity of the natural antimicrobial substance (1%) against MRSA is sustained for 3 months.

In the present disclosure, “natural antimicrobial substance (n %)” means that the natural antimicrobial substance of the present disclosure is dissolved in multi-photocatalyst water or hypochlorous acid water (pH 5) at a concentration of n %.

Antimicrobial Efficacy Test 4—Elimination of Staphylococcus aureus

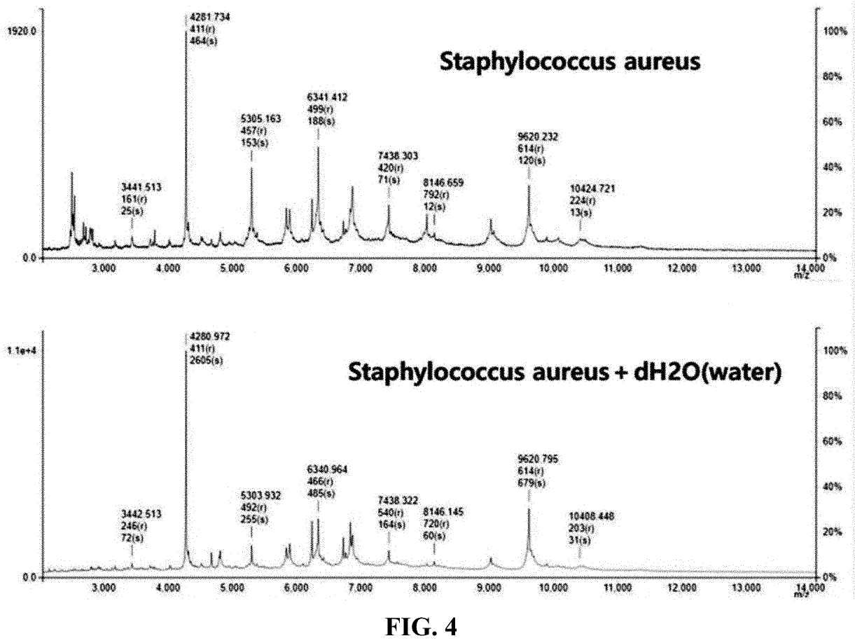

FIGS. 4 to 9 show the data obtained by identifying Staphylococcus aureus and Staphylococcus aureus plus specific substance using a mass spectrometer.

Referring to FIG. 4, it can be seen that, when distilled water was added to Staphylococcus aureus, the mass spectrum of Staphylococcus aureus was maintained and Staphylococcus aureus was identified, indicating that Staphylococcus aureus was not eliminated by distilled water.

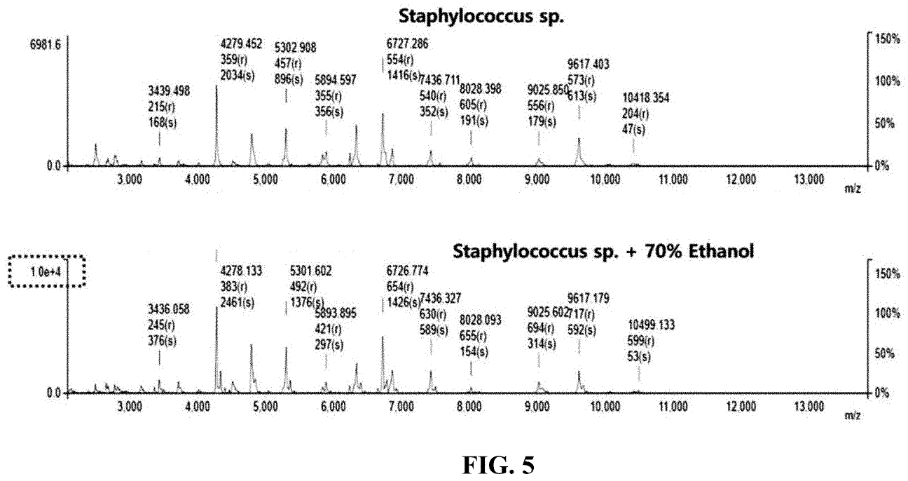

FIG. 5 shows the data obtained by identifying Staphylococcus aureus and Staphylococcus aureus plus 70% ethanol using a mass spectrometer. Referring to FIG. 5, it can be seen that, when 70% ethanol was added to Staphylococcus aureus, the mass spectrum of Staphylococcus aureus was maintained and Staphylococcus aureus was identified, indicating that Staphylococcus aureus was not eliminated by 70% ethanol water.

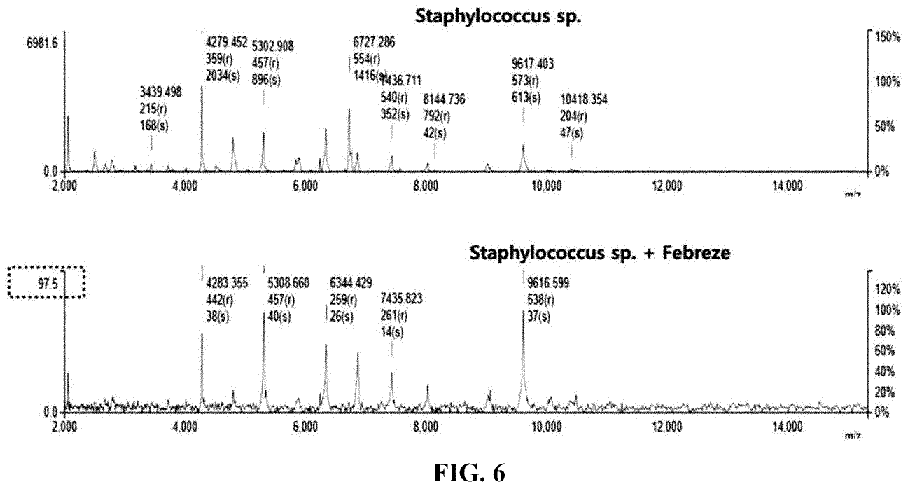

FIG. 6 shows the data obtained by identifying Staphylococcus aureus and Staphylococcus aureus plus Febreze™ using a mass spectrometer. Referring to FIG. 6, it can be seen that, when Febreze™ was added to Staphylococcus aureus, a portion of the mass spectrum of Staphylococcus aureus was lost and changed, so that bacterial identification failed, indicating that Staphylococcus aureus was eliminated by Febreze™.

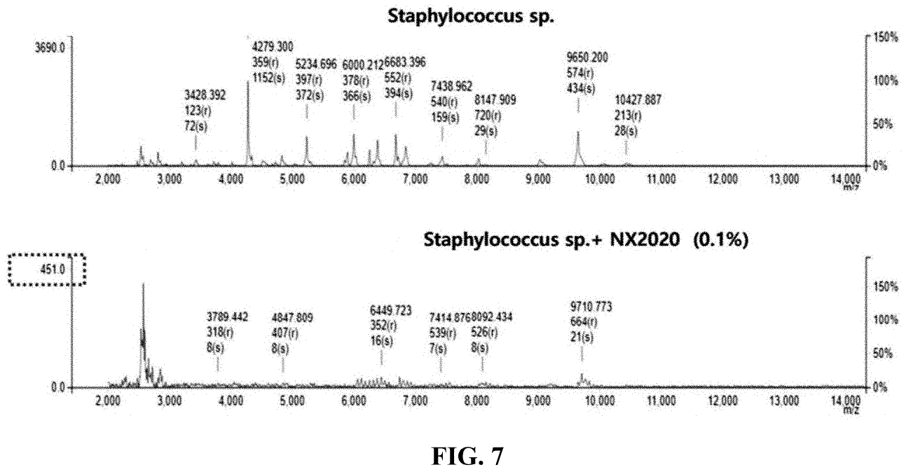

FIG. 7 shows the data obtained by identifying Staphylococcus aureus and Staphylococcus aureus plus the natural antimicrobial substance (0.1%) of the present disclosure using a mass spectrometer. Referring to FIG. 7, it can be seen that, when the natural antimicrobial substance (0.1%) of the present disclosure was added to Staphylococcus aureus, a portion of the mass spectrum of Staphylococcus aureus was lost and changed, so that bacterial identification failed, and the signal intensity also greatly decreased from 3,690 to 451, indicating that Staphylococcus aureus was eliminated by the natural antimicrobial substance (0.1%) of the present disclosure.

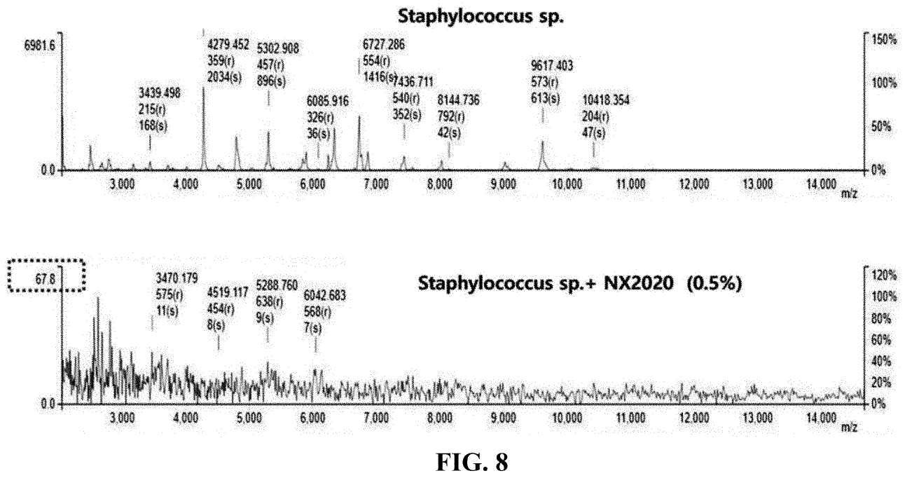

FIG. 8 shows the data obtained by identifying Staphylococcus aureus and Staphylococcus aureus plus the natural antimicrobial substance (0.5%) of the present disclosure using a mass spectrometer. Referring to FIG. 8, it can be seen that, when the natural antimicrobial substance (0.5%) of the present disclosure was added to Staphylococcus aureus, a portion of the mass spectrum of Staphylococcus aureus was changed, so that bacterial identification failed, and the signal intensity also greatly decreased from 6,981.6 to 67.8, indicating that Staphylococcus aureus was eliminated by the natural antimicrobial substance (0.5%) of the present disclosure.

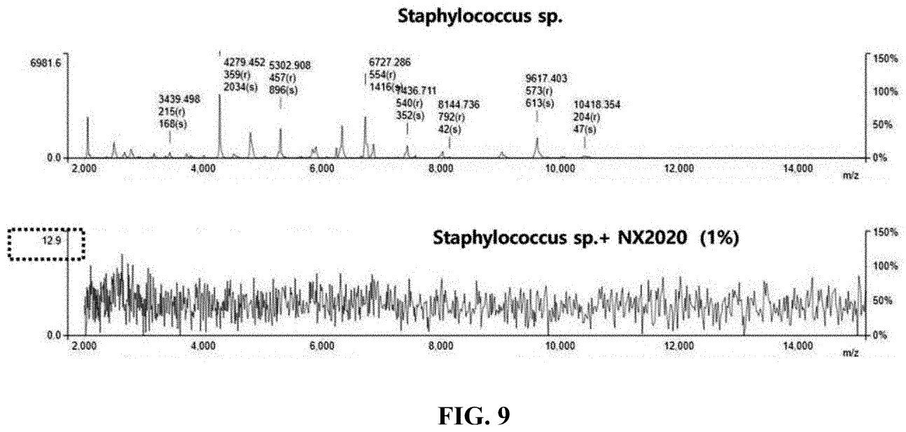

FIG. 9 shows the data obtained by identifying Staphylococcus aureus and Staphylococcus aureus plus the natural antimicrobial substance (1%) of the present disclosure using a mass spectrometer. Referring to FIG. 9, it can be seen that, when the natural antimicrobial substance (1%) of the present disclosure was added to Staphylococcus aureus, the mass spectrum of Staphylococcus aureus was changed, so that bacterial identification failed, and the intensity also greatly decreased from 6,981.6 to 12.9, indicating that the largest amount of Staphylococcus aureus was eliminated by the natural antimicrobial substance (1%) of the present disclosure.

Antimicrobial Efficacy Test 5—Elimination of Pseudomonas aeruginosa

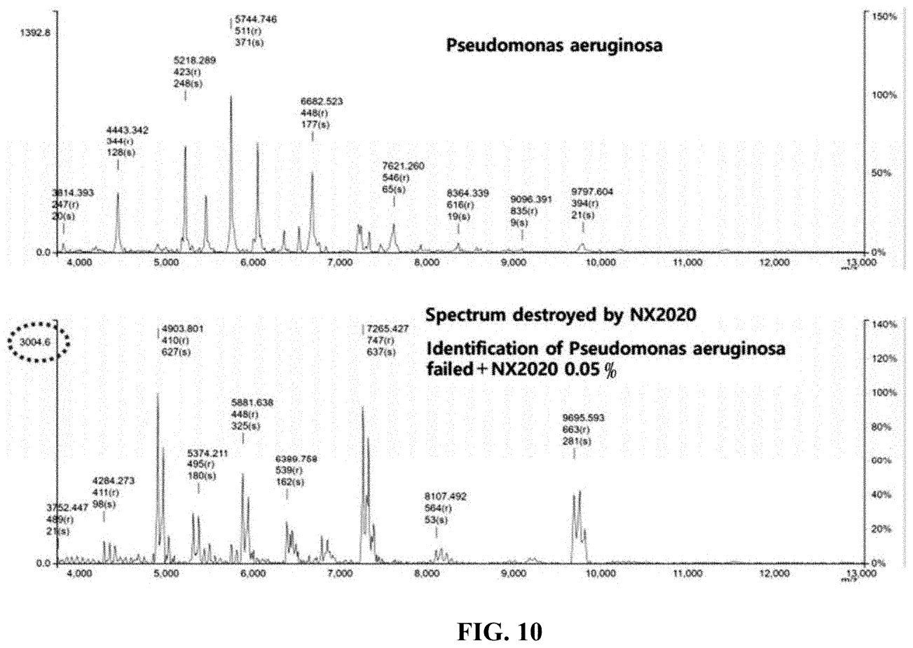

FIG. 10 shows the data obtained by identifying Pseudomonas aeruginosa and

Pseudomonas aeruginosa plus the natural antimicrobial substance (0.05%) of the present disclosure using a mass spectrometer. Referring to FIG. 10, it can be seen that, when the natural antimicrobial substance (0.05%) of the present disclosure was added to Pseudomonas aeruginosa, the intensity increased from 1,392.8 to 3004.6, but the mass spectrum of Pseudomonas aeruginosa was changed, so that bacterial identification failed, indicating that Pseudomonas aeruginosa was eliminated by the natural antimicrobial substance (0.05%) of the present disclosure.

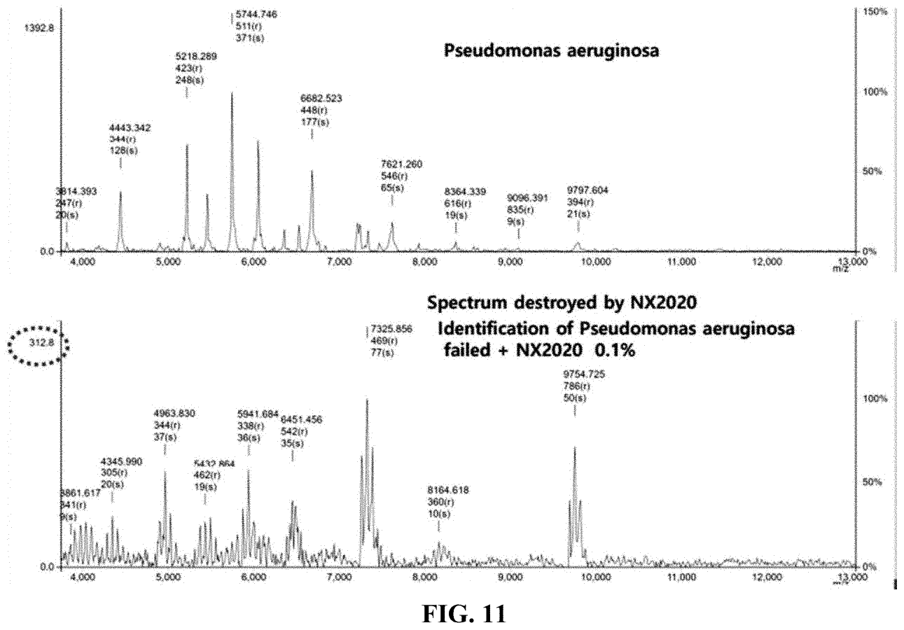

FIG. 11 shows the data obtained by identifying Pseudomonas aeruginosa and Pseudomonas aeruginosa plus the natural antimicrobial substance (0.1%) of the present disclosure using a mass spectrometer. Referring to FIG. 11, it can be seen that, when the natural antimicrobial substance (0.1%) of the present disclosure was added to Pseudomonas aeruginosa, the mass spectrum of Pseudomonas aeruginosa was changed, so that bacterial identification failed, and the intensity also decreased from 1,392.8 to 312.8, indicating that Pseudomonas aeruginosa was eliminated by the natural antimicrobial substance (0.1%) of the present disclosure.

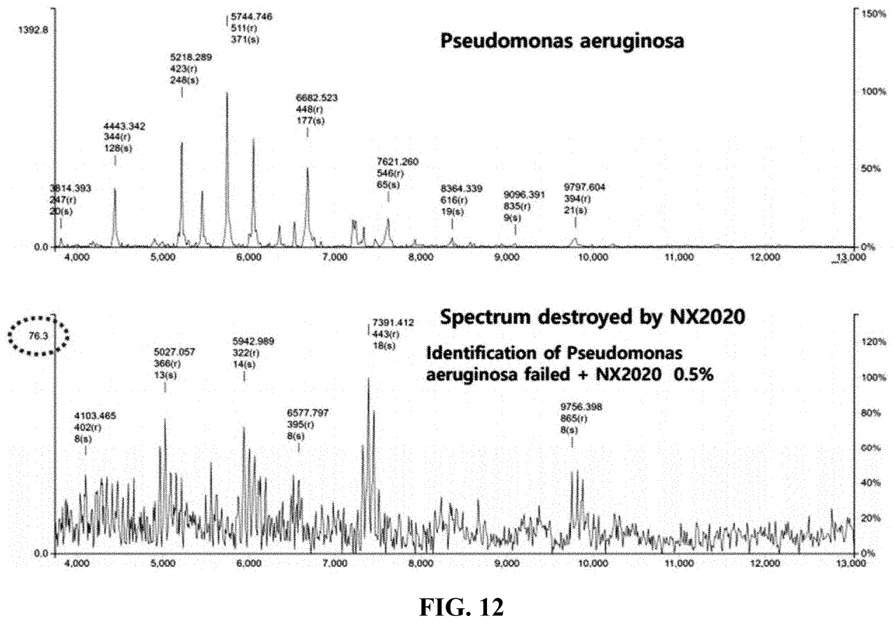

FIG. 12 shows the data obtained by identifying Pseudomonas aeruginosa and Pseudomonas aeruginosa plus the natural antimicrobial substance (0.5%) of the present disclosure using a mass spectrometer. Referring to FIG. 12, it can be seen that, when the natural antimicrobial substance (0.5%) of the present disclosure was added to Pseudomonas aeruginosa, the mass spectrum of Pseudomonas aeruginosa was changed, so that bacterial identification failed, and the intensity also decreased from 1,392.8 to 76.3, indicating that Pseudomonas aeruginosa was eliminated by the natural antimicrobial substance (0.5%) of the present disclosure.

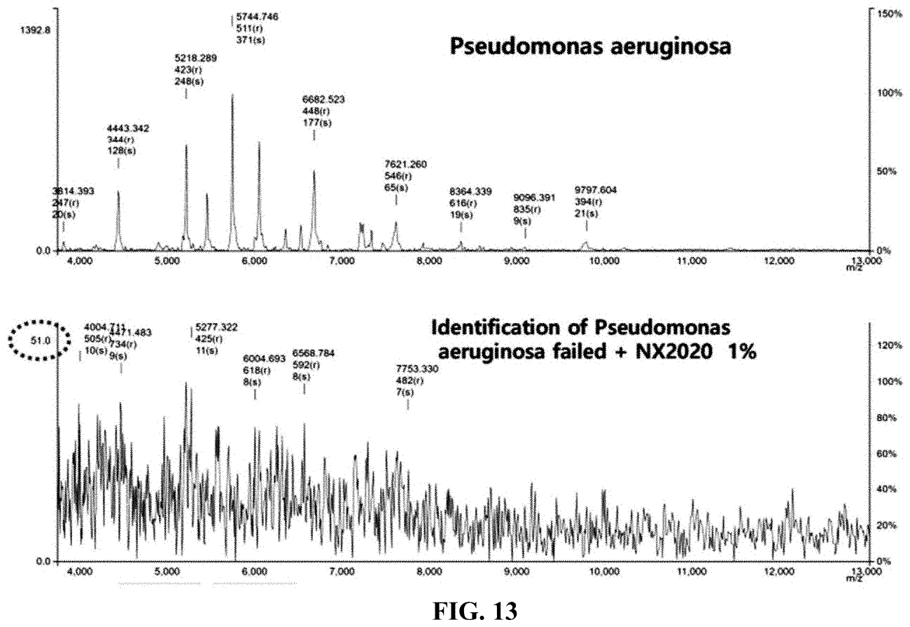

FIG. 13 shows the data obtained by identifying Pseudomonas aeruginosa and Pseudomonas aeruginosa plus the natural antimicrobial substance (1%) of the present disclosure using a mass spectrometer. Referring to FIG. 13, it can be seen that, when the natural antimicrobial substance (1%) of the present disclosure was added to Pseudomonas aeruginosa, the mass spectrum of Pseudomonas aeruginosa was changed, so that bacterial identification failed, and the intensity also decreased from 1,392.8 to 51, indicating that Pseudomonas aeruginosa was best eliminated by the natural antimicrobial substance (1%) of the present disclosure.

Disinfectant for Non-Human Use

A disinfectant for non-human use may be produced by adding the antimicrobial composition comprising the above-described natural antimicrobial substance to multi-photocatalyst water or hypochlorous acid water (pH 2.7 to 6.5) at a concentration of 0.5 to 5%. The disinfectant for non-human use may be used to ensure perfect antimicrobial efficacy in pharmaceutical manufacturing facilities, cosmetics manufacturing facilities, and food manufacturing facilities.

Disinfectant for Human Use

A disinfectant for human use may be produced by adding the antimicrobial composition comprising the above-described natural antimicrobial substance to multi-photocatalyst water or hypochlorous acid water (pH 2.7 to 6.5) at a concentration of 0.1 to 0.5%. The disinfectant for human use has an advantage in that it can minimize skin irritation by replacing conventional disinfectants for human use containing 70% ethanol, thus preventing contact dermatitis.

At this time, if the antimicrobial composition containing the above-described natural antimicrobial substance is added at a concentration of less than 0.1%, the disinfectant effect thereof may be insignificant, and if the antimicrobial composition is added at a concentration exceeding 0.5%, it may cause skin irritation.

The disinfectant for human use may include at least one of a hand sanitizer gel, a hand sanitizer spray, cosmetics, shampoo, toothpaste, a contact lens cleaner, and antibacterial hand wipes.

As described above, the natural antimicrobial substance according to one embodiment of the present disclosure has advantages in that it is safe for human cell membranes and, at the same time, exhibits maximized antibacterial, antifungal and antiviral efficacy by disrupting the cell membrane structure of bacteria.

What is claimed is:

1. A method for producing a natural antimicrobial substance, the method comprising steps of:
adding an organic solvent to each of a Foeniculum vulgare extract aqueous solution and a Polygonum tinctorium extract aqueous solution to obtain a Foeniculum vulgare extract fraction and a Polygonum tinctorium extract fraction;
mixing the Foeniculum vulgare extract fraction and the Polygonum tinctorium extract fraction to obtain a Foeniculum vulgare extract fraction/Polygonum tinctorium extract fraction mixture;
applying ultrasonic stimulation to the obtained Foeniculum vulgare extract fraction/Polygonum tinctorium extract fraction mixture to obtain a nano-sized Foeniculum vulgare extract fraction/Polygonum tinctorium extract fraction mixture; and
applying electrical stimulation to the nano-sized Foeniculum vulgare extract fraction/Polygonum tinctorium extract fraction mixture.

2. The method of claim 1, wherein a weight ratio between the Foeniculum vulgare extract aqueous solution, the Polygonum tinctorium extract aqueous solution and the organic solvent is 1:1.5:7 to 1:2:8.

3. An antimicrobial composition comprising: a natural antimicrobial substance produced by the method of claim 1; and a sugar alcohol.

4. The antimicrobial composition of claim 3, comprising 10 to 30 parts by weight of the Foeniculum vulgare extract, 20 to 30 parts by weight of the Polygonum tinctorium extract, 10 to 20 parts by weight of sorbitol, and 20 to 40 parts by weight of maltitol.

5. A disinfectant for non-human use obtained by adding the antimicrobial composition of claim 3 to multi-photocatalyst water or hypochlorous acid water (pH 2.7 to 6.5) at a concentration of 0.1 to 5%.

6. A disinfectant for human use obtained by adding the antimicrobial composition of claim 3 to multi-photocatalyst water or hypochlorous acid water (pH 2.7 to 6.5) at a concentration of 0.1 to 0.5%.

7. An antimicrobial composition comprising: a natural antimicrobial substance produced by the method of claim 2; and a sugar alcohol.

8. A disinfectant for non-human use obtained by adding the antimicrobial composition of claim 4 to multi-photocatalyst water or hypochlorous acid water (pH 2.7 to 6.5) at a concentration of 0.1 to 5%.

9. A disinfectant for human use obtained by adding the antimicrobial composition of claim 4 to multi-photocatalyst water or hypochlorous acid water (pH 2.7 to 6.5) at a concentration of 0.1 to 0.5%.
